(19) United States
(12) Patent Application Publication
(10) Pub. No.: US 2026/0083470 A1
(43) Pub. Date: Mar. 26, 2026

(54) ORAL STONE SUCTION APPARATUS

(71) Applicant: SHENZHEN HUILAIMEI TECHNOLOGY CO., LTD., SHENZHEN (CN)

(72) Inventors: ZHIHAO YANG, SHENZHEN (CN); WENFENG HUANG, SHENZHEN (CN)

(73) Assignee: SHENZHEN HUILAIMEI TECHNOLOGY CO., LTD., SHENZHEN (CN)

(21) Appl. No.: 18/974,789

(22) Filed: Dec. 10, 2024

(30) Foreign Application Priority Data
Sep. 26, 2024 (CN) 20242238395.6

(51) Int. Cl. A61B 17/24; A61B 1/00; A61B 1/05; A61B 1/06; A61B 1/24; A61B 17/22

(52) U.S. Cl. CPC A61B 17/24; A61B 1/00094; A61B 1/053; A61B 1/0676; A61B 1/0684; A61B 1/00016; A61B 1/00105; A61B 1/24; A61B 2017/22079

(57) ABSTRACT

An oral stone suction apparatus, including a main body, a connecting portion and a suction nozzle portion, where the connecting portion is connected between the main body and the suction nozzle portion. A vacuum-generating device connected to the suction nozzle portion via a hollow pipe is mounted in the cavity of the main body. The suction nozzle portion includes a suction nozzle, an assembly bracket and a suction nozzle pipe housing connected in sequence. The interior of the housing is through in the front-back direction thereof, and the outer diameter of the housing is gradually retracted into a trumpet shape and connected to the suction nozzle via the through pipe. The assembly bracket is provided with at least one mounting hole for mounting a lighting lamp or a camera, and the color thereof is one or more. The oral stone suction apparatus is more efficient, accurate and safe.

### TECHNICAL FIELD

The present disclosure relates to the technical field of oral stone suction, and in particular to an oral stone suction apparatus.

### BACKGROUND

Oral tonsil stones form in the tonsil crypts. The tonsil is lymphatic tissue located in the throat, with many small depressions on the surface called crypts. The accumulation of food particles, bacteria, mucus, and other cellular metabolites within these crypts can lead to the gradual calcification of these materials, resulting in the formation of hard tonsil stones.

The conventional method for removing oral tonsil stones involves using clamping tools, which are maneuvered around the tonsils to dislodge and extract the stones. This process typically requires applying pressure to facilitate the expulsion of the stones from the tonsils.

However, the dim lighting conditions within the oral cavity necessitate the use of auxiliary lighting equipment or the assistance of personnel to provide adequate illumination during the procedure. Even with effective supplemental lighting, identifying the stones can be challenging due to the constricted spaces within the oral cavity. Consequently, the success of stone removal often relies heavily on the clinician's experience. This method frequently results in difficulties in quickly and accurately locating the stones, as well as in employing appropriate techniques for their safe removal. The existing treatment approach predominantly relies on a squeezing mechanism, which, while covering a broad area, significantly compromises precision, thereby rendering the stone removal process highly inconvenient.

### SUMMARY

In order to solve the problem mentioned in the background art, an oral stone suction apparatus is provided.

To solve the technical problems above, the present disclosure provides an oral stone suction apparatus, which includes a main body, a connecting portion and a suction nozzle portion, and the connecting portion is connected between the main body and the suction nozzle portion, where
a vacuum-generating device connected to the suction nozzle portion via a hollow pipe of the connecting portion is mounted in the cavity of the main body;
the suction nozzle portion includes a suction nozzle, an assembly bracket and a suction nozzle pipe housing connected in sequence; the interior of the suction nozzle pipe housing is through in the front-back direction thereof; the outer diameter of the suction nozzle pipe housing is gradually retracted into a trumpet shape on the side thereof close to the suction nozzle, and is communicated with the suction nozzle via a through pipe of the assembly bracket in a horizontal direction; at least one mounting hole for mounting a lighting lamp or a camera is provided on the assembly bracket, and the color of the lighting lamp in the mounting hole is one or more.

Further, the connecting portion includes a connecting disc, and a connecting boss is protruded outwardly from the middle of the front end of the connecting disc; at least one group of L-shaped clamping grooves are provided on the outer wall of the connecting boss; the middle part of the connecting boss is hollowed out to form a stone storage chamber filled with a sponge pad, and the bottom plate of the stone storage chamber is provided with a first through hole backwardly communicated with the main body where the vacuum-generating device is located;
a groove matching the connecting boss is provided on the rear end cover of the suction nozzle pipe housing, and an L-shaped limiting protrusion that can be rotatably clamped to the L-shaped clamping groove is provided on the inner wall of the groove; when the groove is combined with the connecting boss, the L-shaped limiting protrusion enters the L-shaped clamping groove at the same time.

Further, at least one contact copper column is embedded along the axial direction of the outer periphery of the connecting boss of the connecting disk, and the contact copper column forms a contacting contact on the front end face of the connecting disk for signal transmission; an elastic copper column corresponding to the contact copper column of the connecting disk is provided on the end face of the suction nozzle pipe housing, and the elastic copper column forms an elastic contact outside the end face of the suction nozzle pipe housing; when the suction nozzle portion is connected to the connecting portion, the elastic contact is in contact with the contacting contact.

Further, the lighting lamp is an LED lamp bead or a ball-head lamp bead; when the lighting lamp is an LED lamp bead, a lens is mounted on the front side of the mounting hole where the LED lamp bead is located.

Further, the assembly bracket is an elliptical plate structure, the mounting hole is located beside the through pipe, and a camera is embedded in the mounting hole.

Further, the camera is independently connected to a PCBA module in the suction nozzle pipe housing via a wire.

Further, the PCBA module includes an image processing module for processing the image or video signal captured by the camera, a wireless communication module for wireless connection with an external device, a power management module for providing a stable power supply for the image processing module and the wireless communication module, a data storage module with a built-in storage memory for temporarily storing image or video data, and a control module for coordinating the image processing module, the wireless communication module, the power management module and the data storage module.

Further, the external device includes a mobile phone or a tablet computer, the wireless communication module comprises Bluetooth or WiFi, and the wireless communication module wirelessly transmits data to the external device via Bluetooth or WiFi.

Further, the mobile phone or the tablet computer is communicated with the control module via the wireless communication module to call images and videos stored in the data storage module.

Further, the vacuum-generating device includes a waterproof bracket mounted in the cavity of the main body, with a vacuum pump and a battery matched and mounted in the cavity of the waterproof bracket; the vacuum pump and the battery are arranged in parallel in the waterproof bracket, and are electrically connected; the vacuum-generating device further includes a main board, and the battery and the vacuum pump are both electrically connected to the main board; the battery provides power for the main board and the vacuum pump, the main board is fixed to the front side of the waterproof bracket and is close to the side of a button mounted in the main body, and the button can trigger the switch on the main board.

Further, the vacuum-generating device includes a charging board with a USB interface, and the USB interface is mounted with a waterproof plug.

Further, the suction nozzle is a straight or an elbow suction nozzle, and the connection between the suction nozzle and the assembly bracket is sealed.

Further, the upper-end portion, used to connect the suction nozzle portion via the connecting portion, of the main body is configured as a bent shape, and the bending angle of the upper-end portion is in the range of 40°-80°.

Further, the head of the suction nozzle is configured as a hard pipe body, and the tail of the suction nozzle is configured as a soft pipe body.

Further, the filling height of the sponge pad is lower than the cavity height in the stone storage chamber, and the diameter of the stone storage cavity is in the range of 10 mm to 20 mm.

Further, the suction nozzle pipe housing is provided with a second through hole in communication with the stone storage chamber in the groove, and the interior of the suction nozzle pipe housing is through in the front-back direction thereof.

Further, the assembly bracket is of an elliptical plate structure, and the through pipe is located in the middle of the assembly bracket; the mounting holes are respectively arranged on both sides of the through pipe, with the lighting lamps respectively mounted in the mounting holes, and power supply wires of the lighting lamps are connected to the elastic copper column.

The assembly bracket is provided with a lighting lamp and/or a camera. The lighting lamp is engineered to deliver adequate illumination, thereby minimizing reliance on supplementary lighting equipment or personnel. A notable aspect is the utilization of a high-brightness LED light source, which guarantees sufficient lighting during operational procedures. Upon activation, the lamp produces an exceptional lighting effect capable of illuminating the entire oral cavity, thereby providing the operator with a shadow-free, comprehensive field of vision. In comparison to other products available in the market, this ensures that every corner of the oral cavity is uniformly and distinctly illuminated. This characteristic positions the product as a unique solution that effectively addresses the requirements for oral lighting, thereby enhancing the accuracy and efficiency of examinations and treatments. Additionally, the incorporation of a camera facilitates real-time video transmission, allowing the operator to gain a clearer understanding of the internal conditions of the oral cavity, including the location of stones and the status of surrounding tissues. Unlike traditional methods that rely on mechanical extrusion, this employs a vacuum suction device that generates a vacuum adsorption effect through the suction nozzle, enabling the direct absorption of stones without the need for pushing or loosening them. This approach enhances precision and minimizes potential damage to adjacent tissues. Furthermore, the straight and elbow suction nozzles allow for the selection of the most appropriate nozzle based on the specific conditions of individual users and their oral structures, thereby improving operational adaptability and convenience. The oral stone suction apparatus of the present disclosure has the following beneficial effects.
1. Enhanced Precision: The incorporation of a camera facilitates the precise identification of the stone, while vacuum suction allows for its direct and accurate extraction.
2. Increased Safety: The minimization of tissue displacement and compression surrounding the tonsils diminishes the likelihood of trauma during the procedure. The utilization of lighting and camera technology contributes to a more meticulous operation, thereby preventing damage to the oral soft tissues.
3. Streamlined Operational Efficiency: The integrated design simplifies the procedural complexity and reduces reliance on supplementary equipment, thereby enhancing the overall operational workflow.
4. Decreased Dependence on Operator Experience: The method lessens the necessity for the operator's experiential knowledge in stone localization and extraction, thereby mitigating operational challenges and enhancing usability.
5. Enhanced Patient Comfort: The implementation of more precise and gentle techniques during stone removal results in a reduction of patient discomfort.
6. Improved Operational Efficiency: The rapid and accurate removal of stones enhances the overall efficiency of the oral care process.

### BRIEF DESCRIPTION OF DRAWINGS

The present disclosure is described in detail below in conjunction with the accompanying drawings to make the above advantages of the present disclosure more clear.

FIG. 1 is an overall schematic diagram of an embodiment of the present disclosure.

FIG. 2 is a schematic diagram of the structure of the vacuum-generating device of the present disclosure.

FIG. 3 is a schematic diagram of the structure of the connection portion without the sponge pad mounted of the present disclosure.

FIG. 4 is a schematic diagram of the structure of the connection portion of the present disclosure.

FIG. 5 is a first three-dimensional schematic diagram of the suction nozzle portion of an embodiment of the present disclosure.

FIG. 6 is a second three-dimensional schematic diagram of the suction nozzle portion of an embodiment of the present disclosure.

FIG. 7 is a schematic diagram of an assembly bracket of an embodiment of the present disclosure.

FIG. 8 is a schematic diagram of another embodiment of the present disclosure in which a camera is embedded in the assembly bracket.

FIG. 9 is a schematic diagram of the suction nozzle portion configured as an elbow nozzle of the present disclosure.

FIG. 10 is a schematic diagram of the suction nozzle portion configured as a straight nozzle of the present disclosure.

### DETAILED DESCRIPTION

The implementation methods of the present disclosure in detail with reference to the accompanying drawings and embodiments will be described below, such that the implementation process of how the present disclosure applies technical means to solve technical problems and achieve technical effects can be fully understood and implemented accordingly. It should be noted that, provided there are no conflicts, the various embodiments of the present disclosure and the various features in the embodiments can be combined in multiple ways, and the technical solutions formed will all fall within the protection scope of the present disclosure.

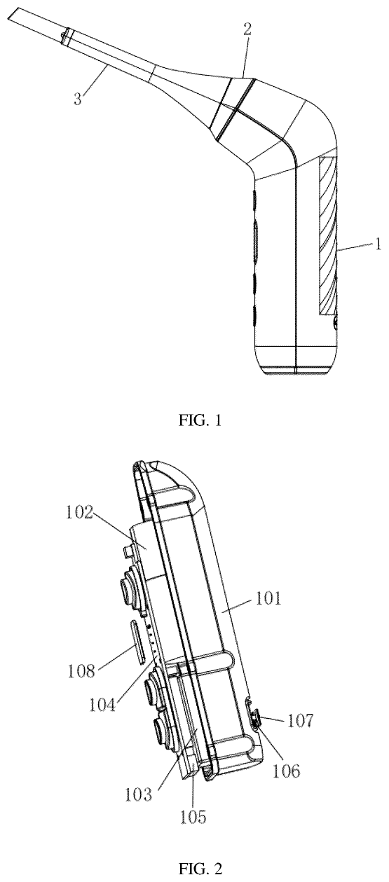

Please refer to FIG. 1. An oral stone suction apparatus is provided, which includes a main body 1, a connecting portion 2 and a suction nozzle portion 3. Structurally, the main body 1 is the handle part of the oral stone suction apparatus, the suction nozzle portion 3 is the part of the oral stone suction apparatus that extends into the oral cavity to absorb stones, and the connecting portion 2 is a component for connecting the main body 1 and the suction nozzle portion 3.

Functionally, a vacuum-generating device is mounted in the cavity of the main body 1, and the vacuum-generating device is connected to the suction nozzle portion 3 through the hollow pipe of the connecting portion 2, so as to realize the vacuum adsorption effect of the suction nozzle portion 3.

The shape of the main body 1 is a cylindrical structure. In other implementations, the shape thereof can be rectangular or elliptical, but it is not limited to the above shapes. Theoretically, any shape that can mount the vacuum-generating device may be used.

The upper-end portion of the main body 1 is bent, and the upper-end portion is used to connect the suction nozzle portion 3 via the connecting portion 2. The bending angle of the upper-end portion is in the range of 40°-80°. Note that this angle is the acute angle between the central axis of the upper-end portion and the axis of the lower handle.

The above vacuum-generating device includes a waterproof bracket 101 (as shown in FIG. 2) mounted in the cavity of the main body 1, and a vacuum pump 102 and a battery 103 are matched and mounted in the cavity of the waterproof bracket 101. It should be understood that the vacuum pump 102 and the battery 103 can be arranged in parallel in the waterproof bracket 101, and the vacuum pump 102 and the battery 103 are electrically connected. The vacuum-generating device also includes a main board 104, and the battery 103 and the vacuum pump 102 are both electrically connected to the main board 104. The battery 103 provides power for the main board 104 and the vacuum pump 102 to ensure the normal operation of the main board 104 and the vacuum pump 102. The main board 104 is fixed to the front side of the waterproof bracket 101 and is close to the side of the button mounted in the main body 1, and the button can trigger the switch on the main board 104. The vacuum-generating device also includes a charging board 105, and the charging board 105 has a USB interface 106, and the USB interface 106 is mounted with a waterproof plug 107 to ensure waterproof performance. The button is embedded in the main body 1 and triggers the switch on the main board 104 by mechanically pressing down, thereby controlling the start and stop of the vacuum pump 102. The main board 104 is electrically connected to the charging board 105, and the charging board 105 is electrically connected to the battery 103. The USB interface 106 on the charging board 105 is used to charge the battery 103. The charging board 105 is connected to an external power source via the USB interface 106 to charge the battery 103. During the charging process, the current is transmitted to the battery 103 via the charging board 105. It should be noted that the charging board 105 is preset with a universal charging circuit, and the main board 104 is preset with a universal control circuit and a driving circuit for driving the vacuum pump 102. The above circuits are designed based on the existing technology, which will not be described here.

Preferably, in this embodiment, a display screen 108 may also be provided on the main body 1, and the display screen 108 is electrically connected to the main board 104. It should be noted that the main board 104 should be preset with a driving circuit or chip for driving the display screen to display the overall working status parameters.

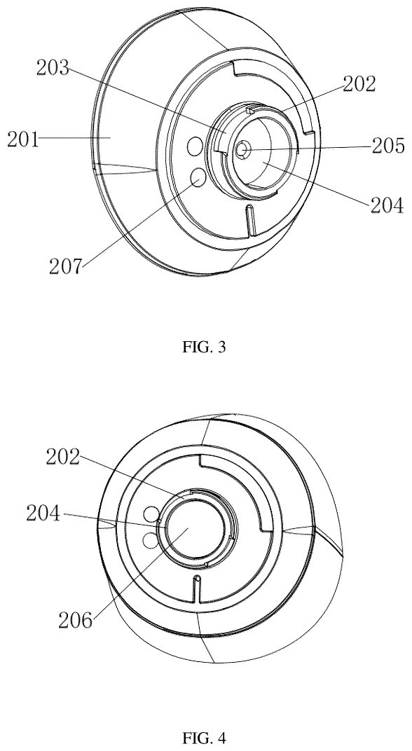

As shown in FIGS. 3 and 4, the connecting portion 2 includes a connecting disc 201, and the rear end of which is matched and connected with the upper-end portion of the main body 1. The matching connection thereof includes a snap connection or a spiral connection or other end face connection methods. A waterproof silicone pad is also provided, which is placed between the end faces of the connecting disc 201 and the upper-end portion of the main body 1 that contact each other, and is used to seal the connection gap between the connecting portion 2 and the main body 1.

A connecting boss 202 is protruded outwardly from the middle of the front end of the connecting disc 201, and at least one group of L-shaped clamping grooves 203 are provided on the outer wall of the connecting boss 202. The middle part of the connecting boss 202 is hollowed out to form a stone storage chamber 204, and the stone storage chamber 204 is filled with a sponge pad 206. The bottom plate of the stone storage chamber 204 is provided with a first through hole 205, and the first through hole 205 is backwardly communicated with the main body 1 where the vacuum-generating device is located. It should be understood that the main body 1 forms a relatively sealed space behind the stone storage chamber 204. When the vacuum pump 102 of the vacuum-generating device is started, the external high-pressure gas enters the main body 1 via the stone storage chamber 204 to form a suction effect. The sponge pad 206 plays the role of filtering impurities. In this embodiment, the impurities are stones. Preferably, in order to provide certain storage space for stones, the filling height of the sponge pad 206 should be lower than the cavity height in the stone storage chamber 204. The diameter of the stone storage cavity 204 ranges from 10 mm to 20 mm, and the number of stones adsorbed ranges from 5 to 10.

At least one contact copper column 207 is embedded along the axial direction of the outer periphery of the connecting boss 202 of the connecting disk 201, and the contact copper column 207 forms two positive and negative contacting contacts on the front end face of the connecting disk 201. At least two contacting contacts in one contact copper column 207 are respectively backwardly connected to the positive and negative electrodes of the battery 103 through wires, and other contact copper columns 207 can be used to transmit data signals. It should be understood that the number of the contact copper columns can be selectively set according to the number of the illumination and camera components of the suction nozzle portion 3.

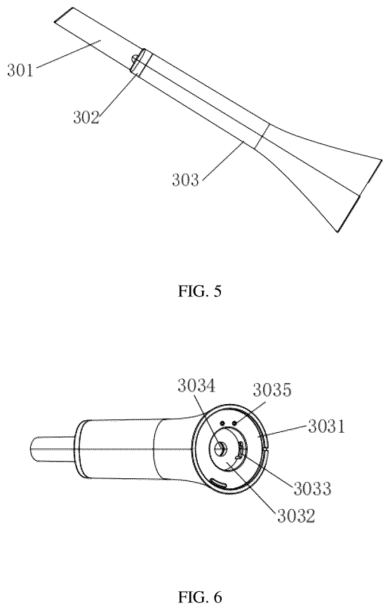

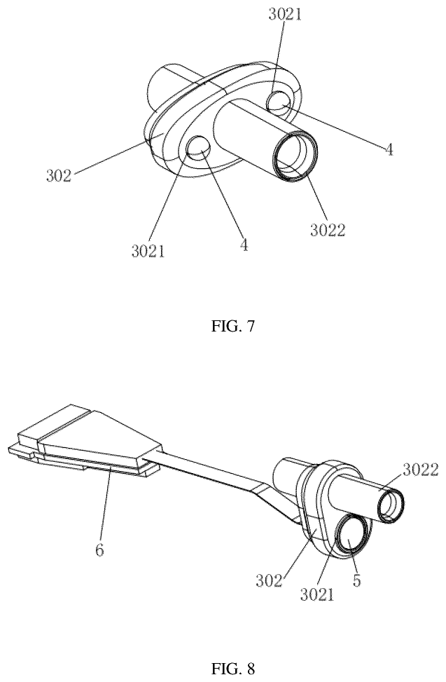

As shown in FIGS. 5 and 6, the suction nozzle portion 3 includes a suction nozzle 301, an assembly bracket 302, and a suction nozzle pipe housing 303 that are connected in sequence. The rear end cover 3031 of the suction nozzle pipe housing 303 is circular, and a groove 3032 matching the connecting boss 202 is provided on the rear end cover 3031. An L-shaped limiting protrusion 3033 that can be rotatably clamped to the L-shaped clamping groove 203 is provided on the inner wall of the groove 3032. When the groove 3032 is combined with the connecting boss 202, the L-shaped limiting protrusion 3033 enters the L-shaped clamping groove 203 (as shown in FIG. 3) at the same time, rotating the suction nozzle pipe housing 303 can make the L-shaped limiting protrusion 3033 match and clamp into the L-shaped clamping groove 203, so as to realize the connection between the suction nozzle portion 3 and the connecting portion 2. In addition, an elastic copper column 3035 corresponding to the contact copper column 207 of the connecting disk 201 is provided on the end face of the suction nozzle pipe housing 303, and the elastic copper column forms an elastic contact outside the end face of the suction nozzle pipe housing 303. When the suction nozzle portion 3 is connected to the connecting portion 2, the elastic contact contacts the contacting contacts to realize the conduction of power source. The suction nozzle pipe housing 303 is provided with a second through hole 3034 in communication with the stone storage chamber 204 in the groove 3032. The interior of the suction nozzle pipe housing 303 is through in the front-back direction thereof. The outer diameter of the housing of the suction nozzle pipe housing 303 is gradually retracted into a trumpet shape on the side thereof close to the suction nozzle 301 and is connected to the suction nozzle 301 in the horizontal direction through the through pipe 3022 of the assembly bracket 302. The assembly bracket 302 is provided with a mounting hole 3021 for mounting a lighting lamp 4 or a camera 5, and the rear end wire of the lighting lamp 4 or the camera 5 is located inside the suction nozzle pipe housing 303. In this embodiment, as shown in FIG. 7, the assembly bracket 302 is an elliptical plate structure, the through pipe 3022 is located in the middle of the assembly bracket 302, and two mounting holes 3021 that are arranged on both sides of the through pipe 3022 are provided. The two mounting holes 3021 are both mounted with a lighting lamp 4, and the lighting lamps 4 can be of the same color or different colors. The power supply wires of the lighting lamps 4 are connected to the elastic copper column. It should be understood that in this embodiment, the suction nozzle 301 is straight. The through pipe 3022 is coaxially arranged with the suction nozzle 301. When the mounting hole 3021 is located beside the through pipe 3022, the mounting position of the lighting lamp 4 is also located beside the suction nozzle 301, and the lighting lamp 4 is located at the side-rear of the suction nozzle 301, such that the moving space of the suction nozzle 301 in front will not be affected, and the vicinity of the suction nozzle 301 can be illuminated by means of the lighting lamp 4 at the nearest position. The above-mentioned lighting lamp 4 can be an LED lamp bead or a ball-head lamp bead. In particular, when an LED lamp bead is used, a lens can be mounted on the front side of the mounting hole 3021 to prevent water seepage. When a ball-head lamp bead is used, due to the function of preventing water seepage thereof, the lens can be omitted.

In another embodiment, as shown in FIG. 8, the mounting bracket has one mounting hole 3021, which is located at the lower side of the through pipe 3022. A camera 5 is matched and embedded in the mounting hole 3021. The camera 5 includes a camera assembly for capturing images or videos. The camera 5 is independently connected to the PCBA module 6 in the suction nozzle pipe housing 303 via a wire. The PCBA module includes an image processing module for processing the image or video signal captured by the camera 5, a wireless communication module for realizing wireless connection with an external device (such as a mobile phone or a tablet computer), including but not limited to Bluetooth or WiFi, a power management module for providing a stable power supply for the image processing module and the wireless communication module, a data storage module with a built-in storage memory for temporarily storing image or video data, and a control module for coordinating the work of each module, which includes but is not limited to a microcontroller or a processor.

When working, the image or video signal captured by the camera module is transmitted to the image processing module through a wire. The image processing module processes the received signal, such as adjusting the resolution, compressing the data, etc. The image processing module transmits the processed image or video data to the wireless communication module, and the wireless communication module transmits the data wirelessly to the external device via Bluetooth or WiFi. The power management module provides the required power for the camera module, the image processing module and the wireless communication module. The power management module includes a battery charging circuit and a circuit for allocating appropriate voltage and current to each module. Before the image or video data is sent to the external device, it is temporarily stored in the storage module. The storage module can be connected to the image processing module and the wireless communication module to transmit data when needed and provide functions such as playback. The control module coordinates the work of each module, such as starting the capture of the camera module, controlling the operation of the image processing module and the data transmission of the wireless communication module. The control module can also receive instructions from external devices, such as starting/stopping video transmission.

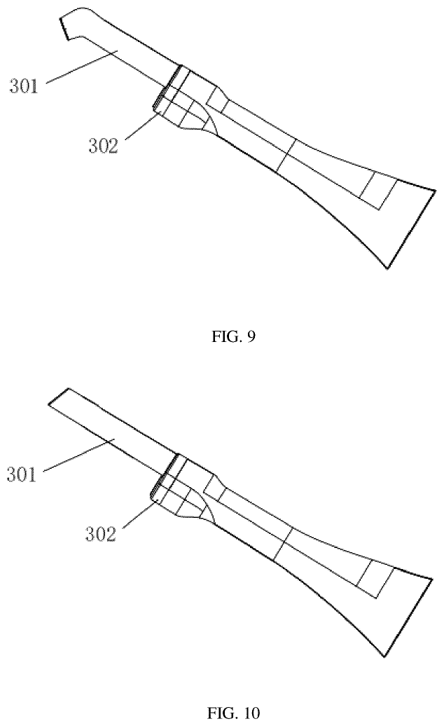

As shown in FIG. 9, in another embodiment, the suction nozzle 301 is an elbow suction nozzle, and the connection between the suction nozzle 301 and the assembly bracket 302 is sealed. In this embodiment, a waterproof ring is mounted between the suction nozzle 301 and the assembly bracket 302 by glue, however, this method is not exclusive. Theoretically, the incorporation of a waterproof pad between the two components, or the application of plastic welding or ultrasonic welding techniques, could also facilitate a sealed connection.

As shown in FIG. 10, in another embodiment, the head of the suction nozzle 301 is configured as a hard pipe body, and the tail of the suction nozzle 301 is configured as a soft pipe body. The hard pipe body acts on the oral stones to facilitate the absorption of the stones, and the soft pipe body provides a buffer force to avoid harming the oral cavity.

It is important to clarify that the above description is only a preferred embodiment of the present disclosure and is not intended to limit the present disclosure. Although the present disclosure is described in detail with reference to the aforementioned embodiments, those skilled in the art can still modify the technical solutions described in the aforementioned embodiments or replace some of the technical features therein by equivalents. Any modification, equivalent replacement, improvement, etc. made within the spirit and principle of the present disclosure shall be included in the protection scope of the present disclosure.

What is claimed is:

1. An oral stone suction apparatus, comprising: a main body, a connecting portion and a suction nozzle portion, and the connecting portion being connected between the main body and the suction nozzle portion, wherein
a vacuum-generating device connected to the suction nozzle portion via a hollow pipe of the connecting portion is mounted in a cavity of the main body;
the suction nozzle portion comprises a suction nozzle, an assembly bracket and a suction nozzle pipe housing connected in sequence; an interior of the suction nozzle pipe housing is through in a front-back direction thereof; an outer diameter of the suction nozzle pipe housing is gradually retracted into a trumpet shape on a side thereof close to the suction nozzle, and is communicated with the suction nozzle via a through pipe of the assembly bracket in a horizontal direction; at least one mounting hole for mounting a lighting lamp or a camera is provided on the assembly bracket, and color of the lighting lamp in the mounting hole is one or more.

2. The oral stone suction apparatus according to claim 1, wherein the connecting portion comprises a connecting disc, and a connecting boss is protruded outwardly from a middle of a front end of the connecting disc; at least one group of L-shaped clamping grooves are provided on an outer wall of the connecting boss; a middle part of the connecting boss is hollowed out to form a stone storage chamber filled with a sponge pad, and a bottom plate of the stone storage chamber is provided with a first through hole backwardly communicated with the main body where the vacuum-generating device is located;
a groove matching the connecting boss is provided on a rear end cover of the suction nozzle pipe housing, and an L-shaped limiting protrusion that can be rotatably clamped to the L-shaped clamping groove is provided on an inner wall of the groove; when the groove is combined with the connecting boss, the L-shaped limiting protrusion enters the L-shaped clamping groove at the same time.

3. The oral stone suction apparatus according to claim 2, wherein at least one contact copper column is embedded along an axial direction of an outer periphery of the connecting boss of the connecting disk, and the contact copper column forms a contacting contact on a front end face of the connecting disk for signal transmission; an elastic copper column corresponding to the contact copper column of the connecting disk is provided on an end face of the suction nozzle pipe housing, and the elastic copper column forms an elastic contact outside the end face of the suction nozzle pipe housing; when the suction nozzle portion is connected to the connecting portion, the elastic contact is in contact with the contacting contact.

4. The oral stone suction apparatus according to claim 1, wherein the lighting lamp is an LED lamp bead or a ball-head lamp bead; when the lighting lamp is an LED lamp bead, a lens is mounted on a front side of the mounting hole where the LED lamp bead is located.

5. The oral stone suction apparatus according to claim 1, wherein the assembly bracket is of an elliptical plate structure, the mounting hole is located beside the through pipe, and a camera is embedded in the mounting hole.

6. The oral stone suction apparatus according to claim 5, wherein the camera is independently connected to a PCBA module in the suction nozzle pipe housing via a wire.

7. The oral stone suction apparatus according to claim 6, wherein the PCBA module comprises an image processing module for processing the image or video signal captured by the camera, a wireless communication module for wireless connection with an external device, a power management module for providing a stable power supply for the image processing module and the wireless communication module, a data storage module with a built-in storage memory for temporarily storing image or video data, and a control module for coordinating the image processing module, the wireless communication module, the power management module and the data storage module.

8. The oral stone suction apparatus according to claim 7, wherein the external device comprises a mobile phone or a tablet computer, the wireless communication module comprises Bluetooth or WiFi, and the wireless communication module wirelessly transmits data to the external device via Bluetooth or WiFi.

9. The oral stone suction apparatus according to claim 8, wherein the mobile phone or the tablet computer is communicated with the control module via the wireless communication module to call images and videos stored in the data storage module.

10. The oral stone suction apparatus according to claim 1, wherein the vacuum-generating device comprises a waterproof bracket mounted in the cavity of the main body, with a vacuum pump and a battery matched and mounted in a cavity of the waterproof bracket; the vacuum pump and the battery are arranged in parallel in the waterproof bracket and are electrically connected; the vacuum-generating device further comprises a main board, and the battery and the vacuum pump are both electrically connected to the main board; the battery provides power for the main board and the vacuum pump, the main board is fixed to a front side of the waterproof bracket and is close to a side of a button mounted in the main body, and the button can trigger a switch on the main board.

11. The oral stone suction apparatus according to claim 10, wherein the vacuum-generating device further comprises a charging board with a USB interface, and the USB interface is mounted with a waterproof plug.

12. The oral stone suction apparatus according to claim 1, wherein the suction nozzle is a straight or an elbow suction nozzle, and connection between the suction nozzle and the assembly bracket is sealed.

13. The oral stone suction apparatus according to claim 1, wherein an upper-end portion, used to connect the suction nozzle portion via the connecting portion, of the main body is configured as a bent shape, and a bending angle of the upper-end portion is in a range of 40°-80°.

14. The oral stone suction apparatus according to claim 12, wherein a head of the suction nozzle is configured as a hard pipe body, and a tail of the suction nozzle is configured as a soft pipe body.

15. The oral stone suction apparatus according to claim 2, wherein a filling height of the sponge pad is lower than a cavity height in the stone storage chamber, and a diameter of the stone storage cavity is in a range of 10 mm to 20 mm.

16. The oral stone suction apparatus according to claim 1, wherein the suction nozzle pipe housing is provided with a second through hole in communication with the stone storage chamber in the groove, and an interior of the suction nozzle pipe housing is through in a front-back direction thereof.

17. The oral stone suction apparatus according to claim 2, wherein the assembly bracket is of an elliptical plate structure, and the through pipe is located in a middle of the assembly bracket; the mounting holes are respectively arranged on both sides of the through pipe, with the lighting lamps respectively mounted in the mounting holes, and power supply wires of the lighting lamps are connected to the elastic copper column.
